(19) United States
(12) Patent Application Publication
(10) Pub. No.: US 2008/0241281 A1
(43) Pub. Date: Oct. 2, 2008

(54) Compositions & methods for activation and inhibition of Staphylococcus aureus biofilm development

(75) Inventors: Govindsamy Vediyappan, Lubbock, TX (US); Revathi Govind, Lubbock, TX (US); Joseph A. Fralick, Lubbock, TX (US)

Correspondence Address: KAMMER BROWNING PLLC, 7700 BROADWAY, SUITE 202, SAN ANTONIO, TX, 78209, US

(73) Assignee: Texas Tech University System, Lubbock, TX (US)

(21) Appl. No.: 11/731,010

(22) Filed: Mar. 29, 2007

(51) Int. Cl. A01N 37/18; A01N 65/00; C07K 1/00; C12N 1/12

(52) U.S. Cl. 424/725; 435/252.1; 514/2; 530/344

(57) ABSTRACT

Botanical extracts and derivative compositions are described for activating or inhibiting the formation and development of bacterial biofilms. Gymnemic acids and other associated saponins are isolated from botanical extractions of the Gymnema sylvestre plant and are used to modulate bacterial biofilm virulence, especially biofilms associated with Staphylococcus aureus. Gurmarin, a polypeptide isolated from botanical extractions of the Gymnema sylvestre plant are used to inhibit biofilm formation, especially biofilms associated with Staphylococcus aureus. Methods for isolating gurmarin (as well as other peptides from botanical extracts) are described using isoelectric focusing separation techniques. Various uses for both research and health care concerns are described for the biofilm activators (modulators) and for the biofilm inhibitor.

### BACKGROUND OF THE INVENTION

1. Field of the Invention

The present invention relates generally to compositions and methods for the activation and inhibition of biofilm development. The present invention relates more specifically to Gymnema sylvestre extracts and derivatives, their use for the activation and inhibition of Staphylococcus aureus biofilm development, and methods for isolating the same.

2. Description of the Related Art

(a) Background on Bacteria and Biofilms in General

Bacteria possess the ability to form aggregated, organized, colonial communities called biofilms. Distinct from their free-living planktonic counterparts, bacterial cells that form biofilms secrete an exopolysaccharide slime that surrounds and protects the bacterial colony. Such biofilm capsules cloak antigenic proteins on the bacterial surface that could otherwise provoke an immune response and thereby lead to the destruction of the bacteria. Capsular polysaccharides are water soluble, commonly acidic, and have molecular weights on the order of 100-1000 kDa. They are generally linear and consist of regularly repeating subunits of one-six monosaccharides. There is however much structural diversity between various types and forms of biofilms. Nearly two hundred different polysaccharides are produced by E. coli alone. Mixtures of capsular polysaccharides, either conjugated or native are sometimes used as vaccines. Bacteria and many other microbes, including fungi and algae, often secrete polysaccharides as an adaptation to help them adhere to surfaces and to prevent them from drying out. Some of these polysaccharides have been developed into useful products, including xanthan gum, dextran, gellan gem, and pullulan. By adhering to each other and to surfaces or interfaces, these matrix-enclosed bacterial populations can cause any number of problems. By attaching to a variety of surfaces such as contact lenses, water pipes, hip replacement implants, and food packaging, they can cause irritation, disease, immune rejection, and food poisoning.

Biofilms are medically and industrially important because they can accumulate on a wide variety of substrates and are resistant to antimicrobial agents and detergents. Microbial biofilms develop when microorganisms adhere to a surface and produce extra-cellular polymers that facilitate adhesion and provide a structural matrix. Therefore, inhibiting microorganism adhesion to surfaces is important. These surfaces of concern may be inert, non-living material or living tissue.

Biofilm-associated microorganisms behave differently from planktonic organisms with respect to growth rates and their ability to resist antimicrobial treatments and host immune defenses and can therefore pose a significant public health problem. Many chronic infections that are difficult or impossible to eliminate with conventional antibiotic therapies are known to involve biofilms. As current antibiotic therapies offer limited effectiveness in treating biofilm infection, a need exists for developing therapeutic agents that prevent or in some way modulate biofilm formation. Polypeptides that regulate biofilm formation provide new compositions that are useful in the diagnosis, treatment, and prevention of bacterial infection and pathogenesis, as well as biofilm formation in both industrial and medical settings.

(b) Background on Staphylococcus aureus

In addition to attaching to abiotic surfaces, many biofilm-forming bacteria colonize living tissue where they can cause serious infection. For example, Staphylococcus aureus, an important human pathogen, is a hardy, gram-positive bacterium that colonizes the skin of most humans. When the skin or mucous membrane barriers are disrupted, staphylococci can cause localized and superficial infections that are commonly harmless and self-limiting. However, when staphylococci invade the lymphatics and the blood, potentially serious complications may result, such as bacteremia, septic shock, and serious metastatic infections, including endocarditis, arthritis, osteomyelitis, pneumonia and abscesses in virtually any organ. Certain strains of S. aureus produce toxins that cause skin rashes, food poisoning, or multi-system dysfunction (as in toxic shock syndrome).

S. aureus is also one of the most common causes of nosocomial infection in U.S. hospitals. It is a frequently isolated pathogen in both primary and secondary bacteremias and in cutaneous and surgical wound infections. Infection by staphylococci usually results from a combination of bacterial virulence factors and a diminution in host defenses. Important microbial factors include the ability of the staphylococcus to survive under harsh conditions, its cell wall constituents, the production of enzymes and toxins that promote tissue invasion, its capacity to persist intra-cellularly in certain phagocytes, and its potential to acquire resistance to antimicrobial agents.

Thus, staphylococci, especially S. aureus, are important pathogenic bacteria causing infections, inflammations and pyosis of human beings and animals. They also produce an enterotoxin which reacts as a pathogen of food poisoning. Recently, methacillin-resistant S. aureus (MRSA) having resistance to many kind of antibiotics, has raised serious problems of nosocomial infections or grave enteritis caused by microbial selection and substitution. Since MRSA shows resistance to almost all antibiotics used practically such as penicillin and methicillin, it infects, for example, postoperative patients with reduced resistance and causes intractable pyosis and inflammation. S. aureus is commonly isolated from bacterial biofilms.

(c) Background on Gymnema sylvestre and Gurmarin

It has long been known that chewing a piece of the leaf of Gymnema sylvestre, used as a folk remedy in India for various afflictions including diabetes mellitus, causes complete loss of sweet taste sensation. The active substance which suppresses sweet taste was first extracted as a mixture of acidic compounds, and has generally been called gymnemic acid. More recently, a new substance referred to as gurmarin has been isolated (with greater difficulty) from the hot water extracts of the leaves of G. sylvestre, and has been shown to suppress the sweet taste responses in rats, although it has little or no effect on the sweet taste response in humans. Gurmarin, as described in detail below, has been studied extensively as a therapeutic composition for the treatment of diabetes.

Gurmarin is a 35 amino acid residue polypeptide that includes three intra-molecular disulfide bonds and has a relatively high molecular weight. Gurmarin can be obtained by chemical synthesis, and also by extraction from the leaves of at least one plant from the genus Gymnema (family Asclepiadaceae). A convenient source of gurmarin as mentioned above is Gymnema sylvestre, a plant that grows principally in Central and Western India, tropical Africa, and Australia. The structure of gurmarin can be represented by the formula shown in FIG. 4.

A series of patents issued to Chatterji (U.S. Pat. No. 6,946,151; U.S. Pat. No. 6,949,261; and U.S. Pat. No. 7,115,284), the full disclosures of which are incorporated herein by reference, have explored the use of gurmarin as a therapeutic composition for the treatment of diabetes and have described certain methods for extracting the compound from G. sylvestre. These prior methods have relied strictly on separation of the peptide according to the high molecular weight of the compound (>3000 Daltons). These techniques have generally involved simple filtration of a prepared solution based on molecular weight. The effectiveness of simple filtration, however, to extract a higher molecular weight compound is highly dependent on the clarity of composition before filtration and/or the ability to further separate the retention. Further, the filtration device used in the earlier art will retain all molecules above 3000 Daltons and therefore may contain a mixture of other large molecules. In order to identify and characterize novel bioactive molecules, purification to a homogenous level is important.

There exists a need therefore, in both the research field and in the commercial application field for compositions that serve to inhibit biofilm formation and compositions that activate (modulate) biofilm formation. These needs are present in both the medical/health care environment and in the industrial environment. There are specific needs that exist with respect to the Staphylococcus aureus bacteria biofilms and the virulence of the biofilm that it forms. It would be beneficial if compositions could be identified that addressed the virulence of S. aureus biofilm and further could be used to facilitate the study of the biofilm and provide specific methods for treatment of surfaces and objects for the purposes of inhibiting biofilm formation. It would be beneficial if such compounds were otherwise non-toxic for the purposes of addressing bacterial biofilm formation on human contact surfaces and in human tissue environments.

### SUMMARY OF THE INVENTION

In fulfillment of the above described needs in both the research field and the health care field, the present invention provides botanical extracts and derivative compositions that may be used for activating or inhibiting the formation and development of bacterial biofilms. All of the compositions are derived from the botanical Gymnema sylvestre. In a first preferred embodiment, gymnemic acids in conjunction with other associated saponins are isolated from botanical extractions of the G. sylvestre plant and are used to modulate bacterial biofilm virulence, especially for biofilms associated with Staphylococcus aureus. Known mechanisms and methods for extracting the gymnemic acids from G. sylvestre are utilized to create compositions that are herein shown to modulate the virulence factors associated with bacterial biofilms.

In contrast to the activating properties of the gymnemic acid compositions, gurmarin, a polypeptide isolated from botanical extractions of the Gymnema sylvestre plant, is used to inhibit biofilm formation, especially biofilms associated with Staphylococcus aureus. Unique methods for isolating gurmarin are described using isoelectric focusing separation techniques. These isoelectric focusing separation methods may be used to isolate a variety of peptides from similar botanical extractions for both analytical use and productive use.

A variety of applications in both the research field and in the health care industry are anticipated for the biofilm activators (modulators) and for the biofilm inhibitor. The biofilm activator (modulator) compounds are most useful for research studies to measure the effectiveness of antimicrobial compounds but may also find application in industrial biofilm formation needs. The biofilm inhibitor compounds are most useful for application in the health care industry as mechanisms for controlling the formation of virulent bacteria strains and their related biofilms.

Further applications of both the biofilm activators (gymnemic acid compositions) and the biofilm inhibitors (gurmarin compositions) will be recognized by those skilled in the art. The above mentioned objectives are fulfilled in the present invention in the implementation of the preferred embodiments of the invention which are described below. These preferred embodiments are not meant to be limiting of the scope of the present invention which is set forth more specifically in the claims that follow.

### BRIEF DESCRIPTION OF THE DRAWINGS

FIG. 1 is an image of a linear gradient sodium dodecyl sulfate polyacrylamide gel electrophoresis (SDS-PAGE) (5%-15%) showing analytical separation of a number of secreted proteins of S. aureus biofilm with (+) and without (−) the presence of the biofilm activator of the present invention.

FIG. 2A is a comparative image of microtiter wells exhibiting a S. aureus biofilm assay with and without the biofilm activator of the present invention.

FIG. 2B is a comparative image of a western blot exhibiting poly N-Acetylglucosamine (PNAG) [a major component of capsular polysaccharide] activity in a S. aureus biofilm with and without the biofilm activator of the present invention.

FIG. 3A is an image of purified biofilm inhibitor (gurmarin) by an isoelectric focusing (IEF) method and its separation on a SDS-PAGE (20%) of the present invention showing its effect on S. aureus biofilm formation. Gurmarin peptides (fractions 15-19) were stained by Coomassie brilliant blue stain. The (+) and (−) symbols indicate biofilm activation and inhibition respectively.

FIG. 3B is a comparative image of a western blot exhibiting poly N-Acetylglucosamine (PNAG) activity in a S. aureus biofilm with a control, with the biofilm inhibitor, with the biofilm activator, and with both the biofilm inhibitor and activator of the present invention. Note the biofilm inhibitor suppresses the production of PNAG even in the presence of activator.

FIG. 4 is a representation of the complete amino acid sequence of the biofilm inhibitor of the present invention (gurmarin).

### DETAILED DESCRIPTION OF THE PREFERRED EMBODIMENTS

### 1. General Overview

The present invention includes: (a) compositions for activating (modulating) biofilm formation (gymnemic acids); (b) a composition for inhibiting biofilm formation (gurmarin); and (c) a novel method for isolating the biofilm inhibitor (gurmarin) from a botanical extract (Gymnema sylvestre). These compositions are shown to have particular efficacy with respect to Staphylococcus aureus bacteria biofilms. The isolation method is applicable to a range of peptides capable of being isolated from botanical extractions.

### 2. Activator (Modulator) Compositions & Effectiveness

The present invention identifies novel small molecule compounds which activate Staphylococcus aureus biofilm development in vitro, specifically, the identification of a S. aureus biofilm activator (referenced herein as “LBBA-1” from “Laks Biotech Biofilm Activator 1”) containing a mixture of gymnemic acids and other saponins. Gymnemic acids are a group of triterpine glycosides that inhibit sweet taste in humans.

The mechanism of biofilm activation in Staphylococcus aureus by LBBA-1 was studied in the development of the present invention using S. aureus DNA microarray technology. LBBA-1 modulates several genes involved in signal transduction and transcriptional regulators thereby activating biofilm formation. In gene regulation, down regulation occurs when a cell which is overly sensitized from a prolonged/enhanced period of reaction begins to degrade the receptors in order to reduce its sensitivity to that molecule and return to normal. This is a locally acting negative feedback mechanism.

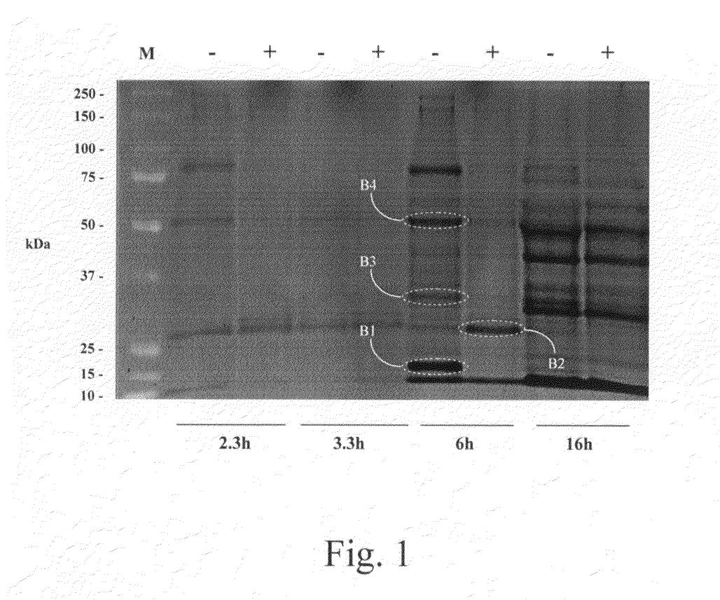

S. aureus produces several extra-cellular virulence factors (proteins). In order to examine the comparative profile of secreted proteins, 1.0 ml of spent growth medium from the indicated biofilms (with or without LBBA-1) at various time points were collected. Trichloroacetic acid (TCA) was precipitated and separated by a linear gradient SDS-PAGE (5-15%). The gel was stained by SYPRO® Ruby (Molecular Probe) and the image was recorded (see FIG. 1) by a phosphoimager (Typhoon 9410, Amersham Biosciences).

Major differences in the secreted protein profile, under above mentioned conditions, were observed only after 6 hours of biofilm growth (see FIG. 1). Selected protein bands (bands B1, B2, B3, and B4) were incised from gel, trypsin digested (Promega) and analyzed by LC-MS/MS (Liquid Chromatograph Tandem Mass Spectrometry). The results are presented in Table 1. Protein bands marked B1, B2, B3, and B4 in FIG. 1 correspond to sample numbers 1-4 in Table 1 (below).

Molecular weights of the proteins determined by SDS-PAGE (see FIG. 1) were matched by predicted protein size in the database which confirmed the identification of secreted biofilm proteins by LC-MS/MS. SasD (Sample No. 1, protein band B1), Alpha Hemolysin α-hla (Sample No. 3, protein band B3) and Immunoglobulin G binding protein precursor (Sample No. 4, protein band B4) were down regulated in LBBA-1 treated biofilms. SAI-B/Immunodominant antigen A (Table 1, Sample No. 2 and FIG. 1, protein band B2) was not down regulated in LBBA-1 treated biofilms.

As shown in Table 1, secreted proteins in the growth medium were separated by linear (5-15%) gradient 1D-SDS-PAGE and SYPRO® stained. Indicated protein bands were incised from the gel and analyzed for LC-MS/MS. The last column in Table 1 represents the apparent molecular weight based on SDS-PAGE. The sequence match column data was based on S. aureus sequences in FSATA, GeneBank databases. The charge column data represents the LC-MS/MS values for a given peptide.

TABLE 1

LC-MS/MS analysis of some secreted proteins of LBBA-1 treated S. aureus
biofilm

| | | | | | |
|---|---|---|---|---|---|
| | | | | | App. |
| Sample | Sequence match | Charge | XC | % aa | MW |

| | | | | |
|---|---|---|---|---|
| 1 | ALPQLSAGSNMQDYNMK | 2 | 4.9 | 11.26 |
| | LYDATQNIADK | 2 | 3.7 | 7.28 |
| | DMNGHALPLTK | 2 | 2.6 | 7.28 |
| | DGNFYQTNVDANGVNHGGSQMVQNK | 2 | 4.0 | 16.56 |

| | | | |
|---|---|---|---|
| Surface protein SasD, predicted MW: 16770 | 42.38 | 27802169 | 16 kDa |

| | | | | |
|---|---|---|---|---|
| 2 | LSNGNTAGATGSSAAQIMAQR | 2 | 3.21 | 9.01 |
| | DGAYDIHFVK | 2 | 3.21 | 4.29 |
| | AQGLGAWGF | 1 | 1.42 | 3.86 |

| | | | |
|---|---|---|---|
| Secretary protein SAI-B, predicted MW: 24214 | 17.17 | 7959131 | 25 kDa |
| Immunodominant antigen A, avg. mass: 24205.0 | | 21284219 | 25 kDa |

| | | | | |
|---|---|---|---|---|
| 3 | VFYSFIDDK | 1 | 1.7 | 3.7 |
| | EYMSTLTYGFNGNVTGDDTGK | 2 | 3.7 | 7.17 |
| | YVQPDFK | 2 | 1.8 | 2.39 |
| | TILESPTDK | 1 | 1.2 | 3.07 |
| | VIFNNMVNQNWGPYDR | 2 | 3.2 | 5.46 |
| | AADNFLDPNK | 2 | 2.2 | 3.41 |
| | ASSLLSSGFSPDFATVITMDR | 2 | 2.8 | 7.17 |
| | DDYQLHWTSTNWK | 2 | 2.5 | 4.44 |

| | | | |
|---|---|---|---|
| Chain B, Alpha-Hemolysin, avg. mass: 33248 | 36.18 | 2914570 | 33 kDa |

| | | | |
|---|---|---|---|
| 4 | NMIKPGQELVVDKKQ | 2 | 2.8 |
| | DDPSQSTNVLGEAKK | 2 | 3.7 |
| | EDGNGVHVVKPGDTVNDIAKA | 2 | 2.8 |

| | | |
|---|---|---|
| Immunoglobulin G binding protein precursor | 21281813 | 50 kDa |

3. Preparation of Activator (Modulator) from Gymnema sylvestre

The following method was used to initially identify the S. aureus biofilm activator (LBBA-1) and its effectiveness. Methanol-water (50:50) extracts were prepared from several ethno-botanical plants and were tested for biofilm modulating properties. One extract was identified and further fractionated on a Sephadex LH-20 column using 60-20% methanol-water gradient to produce what was considered partially purified fractions. Sephadex sub-fractions (SSF) were concentrated and used for S. aureus biofilm development and assay.

In the S. aureus biofilm assay, polystyrene microtiter wells and glass tubes well known in the art were used to develop biofilm in vitro. Fractions dissolved in water were added to a sterile tryptic soy broth (TSB, Difco, Detroit) medium at a final concentration between 0.001-0.01% (w/v). Biofilm was developed and measured according to methods well known in the art. Briefly, a single colony of S. aureus (NCTC-8325-4) (or a clinical isolate) grown on blood agar plate was inoculated into the TSB liquid medium and grown in a shaking water bath at 37° C. until it reached an optical density (OD₆₀₀) of 0.6-0.8. An aliquot was further diluted in TSB to an OD of 0.05 and 0.1 ml was dispensed into sterile microtiter wells. Solubilized SSF were added to the wells (1 μl/ml from 0.1% of filter sterile stock in water). Cells without SSF served as a control. The microtiter plate was incubated at 37° C. with gentle shaking for up to 16 hours.

At different time points, cells were collected and assayed for biofilm growth. Unbound cells were aspirated and saved for measuring the growth at OD₆₀₀ and cell viability on agar plates. Loosely bound cells were removed by washing with phosphate buffer saline (PBS) twice and adherent biofilm cells were stained with 1% crystal violet (CV) stain. After extensive washing, stained cells were air dried, solubilized in dimethylsulfoxide (DMSO) and lysates (100 μl) were measured by a plate reader at OD₅₉₀.

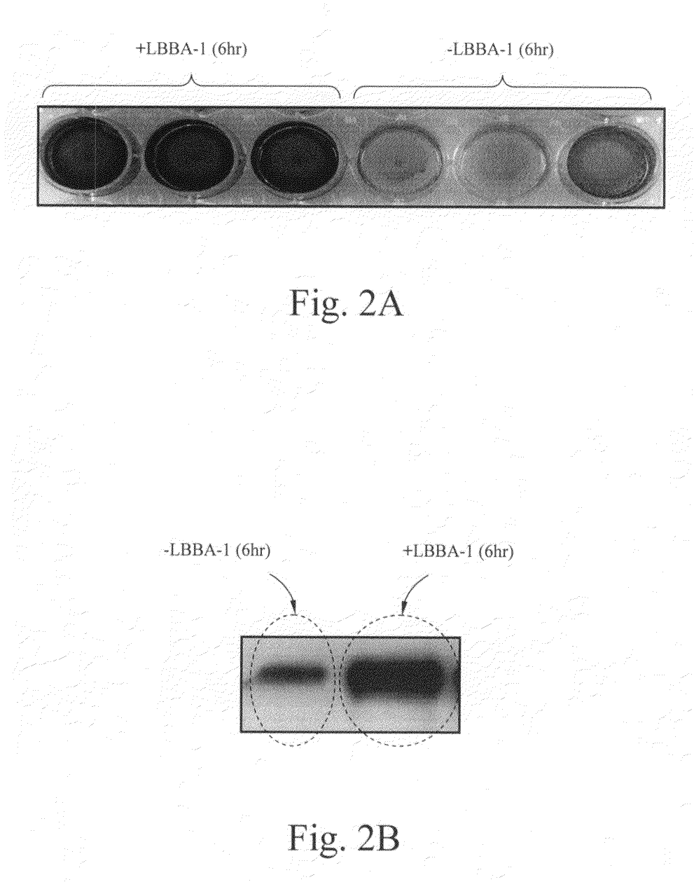

Among several SSF, one fraction contained biofilm activator (LBBA-1) and promoted rapid biofilm formation in a concentration dependent manner (within 2-6 hours) by S. aureus NCTC-8325-4 (as shown in FIG. 2A) or clinical strains (MRSA) ‘in vitro’ without affecting growth or cell viability. Quantization of CV stained biofilms indicated that biofilms from LBBA-1 treated cultures contained approximately 15 times more biofilm material than did control cultures. These biofilm cells were more resistant to vancomycin in comparison to those biofilms formed in the absence of LBBA-1 (control). S. aureus growth in TSB, with or without LBBA-1, is similar, indicating that LBBA-1 does not affect cell growth or its viability. Biofilms developed by S. aureus in the presence of LBBA-1 produced more poly N-acetylglucosamine (PNAG) as determined by western blot (as shown in FIG. 2B) using anti-PNAG rabbit polyclonal antibody. The biofilm activator also modulated some of the biofilm specific gene products (HasD, α-Hla, & IgG binding protein) when compared to control biofilms as determined by proteomic and mass-spectrometry methods (see Table 1). The LBBA-1 substances are dialyzable and proteinase-K insensitive suggesting that they may contain non-proteinaceous small molecules.

Biofilm specific LBBA-1 (gymnemic acid) may therefore be a very useful investigative tool to study S. aureus biofilm biology. As described, the compounds originate from Gymnema sylvestre, a woody climbing plant native to India, Africa, and China. This is a traditional Indian medicinal plant, for which safe use in humans has been demonstrated by several studies and numerous patents (U.S. Pat. Nos. 7,115,284; 5,980,902; 5,900,240; 5,730,988; 5,612,039; 4,912,089; and 4,761,286 for example). Compounds from this plant have been used in the treatment of diabetes, impaired glucose tolerance, and various conditions associated with diabetes. No significant adverse effects have been reported, aside from the expected hypoglycemia associated with the use of Gymnema extracts.

### 4. Inhibitor Composition & Effectiveness

From the same botanical source (Gymnema sylvestre) that provides the biofilm activator compositions described above, a biofilm inhibitor has also been isolated which shows effectiveness against biofilm virulence factors associated with Staphylococcus aureus bacteria biofilms. The biofilm inhibitor of the present invention (identified “LBBI-1” herein from “Laks Biotech Biofilm Inhibitor-1”) comprises the compound gurmarin, especially as extracted from G. sylvestre according to a novel method for isolating the same.

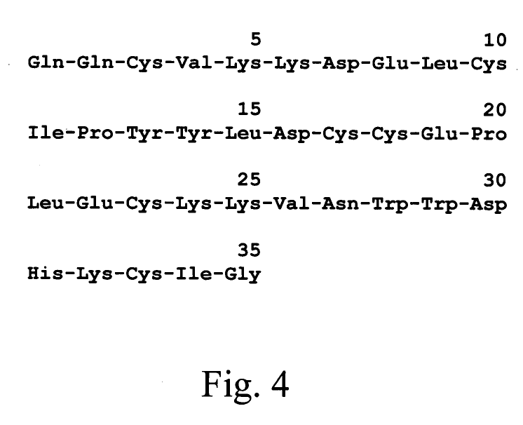

The novel compound (LBBI-1) which inhibits S. aureus biofilm formation was identified and partially purified using a one step effective method for isolation of the compound from a complex botanical extract. Gurmarin, an electrophoretically purified polypeptide isolated from botanical extractions of the Gymnema sylvestre plant, is used in the present invention to inhibit biofilm formation, especially biofilms associated with Staphylococcus aureus. Gurmarin is a 35-residue polypeptide which migrates with an apparent molecular weight of 4-5 kDa on SDS-PAGE as known in the art. The complete amino acid sequence of gurmarin is shown in FIG. 4. High resolution structures of both native gurmarin and synthetic gurmarin have been defined in the art. Gurmarin includes six cystine residues which allow the peptide to attach chemically to the surfaces of several biological and non-biological supports. Furthermore, the biological properties of gurmarin can be increased by shuffling or modifying the amino acids by recombinant and/or synthetic methods. The crystal structures of both native and synthetic gurmarin are available and were found to be similar. Gurmarin is known in the art to suppress the response of the rat chorda tympani nerve to sweet-taste stimuli such as glucose, sucrose, glycine and saccharine without affecting responses to other taste stimuli including NaCl (salty), HCl (sour), and quinine (bitter). Gurmarin has no apparent effect in sweet taste suppression in humans.

5. Isolation of Inhibitor from Gymnema sylvestre Extracts

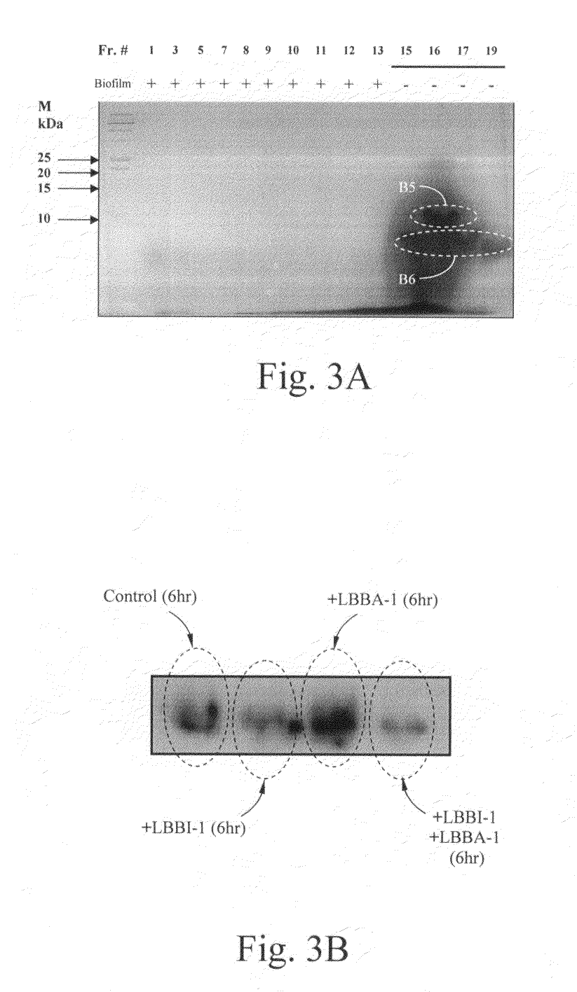

To isolate LBBI-1 (gurmarin) from the complex botanical extract, a preparative isoelectric focusing (IEF) technique which separates peptides based on their isoelectric point pI values was used. IEF involves use of high voltage (1000-3000) and carrier ampholyte which, under the electric field, forms a pH gradient. In the present invention, LBBI-1 (gurmarin) was purified entirely using liquid based medium (ampholyte+water+Gymnema sylvestre extract). This way, it is easy to perform in large amount and yields an effective purified LBBI-1. The electro-focused mixture (whole) can be collected into 20 separate fractions (FIG. 3A). The active fractions containing LBBI-1 were pooled and dialyzed or desalted to remove ampholytes prior to use. The peptides migrate and separate based on pI values. Isoelectric focusing, also known as electro-focusing, is a technique for separating different molecules by their electric charge differences. It is a type of zone electrophoresis, usually performed in a gel, which takes advantage of the fact that a molecule's charge changes with the pH of its surroundings. Molecules are distributed over a medium that has a pH gradient (usually created by aliphatic ampholytes). An electric current is passed through the medium, creating a “positive” anode and “negative” cathode end. Negatively charged particles migrate through the pH gradient toward the “positive” end while positively charged particles move toward the “negative” end. As a particle moves towards the pole opposite of its charge it moves through the changing pH gradient until it reaches a point in which the pH of that molecule's isoelectric point is reached. At this point the molecule no longer has a net electric charge (due to the protonation or deprotonation of the associated functional groups) and as such will not proceed any further within the gel. The gradient is initially established before adding the particles of interest by first subjecting a solution of small molecules such as polyampholytes with varying pI values to electrophoresis.

The method may be applied particularly in the study of proteins, which will separate based on their relative content of acidic and basic residues, whose value is represented by the pI. Proteins are introduced into a gel composed of polyacrylamide, starch, or agarose, where the pH gradient has been established. Gels with large pores are usually used in this process to eliminate any “sieving” effects, or artifacts in the pI caused by differing migration rates for proteins of differing sizes. Isoelectric focusing can resolve proteins that differ in pI value by as little as 0.01. Isoelectric focusing may be the first step in two-dimensional gel electrophoresis, in which proteins are subsequently separated by molecular weight through SDS-PAGE.

This simple but efficient method of purifying small peptides from a complex natural product is advantageous for large scale preparation. This technique can be applied to several other natural products to isolate therapeutic peptides. FIG. 3A demonstrates the migration of isolated LBBI-1 on 20% SDS-PAGE gel with an apparent molecular weight of 4-5 kDa band so as visualized by Coomassie brilliant blue stain. Fractions 15-19 (FIG. 3A) were pooled (LBBI-1) and analyzed for biofilm inhibitory activity.

The pooled LBBI-1 contained a small substance which is dialyzable (10,000 MWCO) and proteinase-K susceptible. The biofilm inhibitory property of LBBI-1 (at 2 μg/ml concentration) was confirmed by microtiter biofilm assay. LBBI-1 did not affect the growth of S. aureus. It is known in the art that an increased amount of PNAG is produced during S. aureus biofilm growth and that PNAG acts as a critical virulence factor in the murine model. In order to determine whether LBBI-1 affects PNAG production, an equal number of 6 hour biofilm cells were taken by adjusting their OD₆₀₀ and used for assaying PNAG by western blot according to a method known in the art.

As shown in FIG. 3B, LBBI-1 prevents S. aureus biofilm growth and shows significantly less PNAG production compared to biofilm grown with activator (compare +LBBA-1 and +LBBI-1). When both LBBI-1 and LBBA-1 were incubated together during biofilm growth, PNAG synthesis is almost abolished (FIG. 3B, see +LBBI-1 +LBBA-1). These results indicate that LBBI-1 can inhibit biofilm production even in the presence of LBBA-1. These results demonstrate that IEF methods are very useful techniques to isolate and purify biologically active peptides such as LBBI-1 from a natural source. LBBI-1 is a specific inhibitor of S. aureus biofilm formation and has potential commercial value in the pharmaceutical sector.

The cost of treating human biofilm-related illness accounts for billions of health care dollars annually in the United States alone. LBBI-1 can be used as an investigative tool in microbial biofilm studies. LBBI-1 can also be used for various therapeutic purposes such as oral biofilm prevention, inhibition of biofilm on wounds (particularly diabetic and burn wounds), as a tool for quorum sensing studies, and as a coating for various medical devices (e.g. catheters, dentures, and stents) to prevent implant-related biofilm.

One of the desirable properties of a therapeutic agent is its non-toxicity to human beings or other animals. Gymnema sylvestre extracts have been used for several diseases, particularly for diabetes. As further support, both LBBA-1 and LBBI-1 were found to be non-toxic to several cell lines (human) tested.

As discussed above, Staphylococcus aureus is a major cause of human disease due to its high human adaptability and toxin producing abilities. S. aureus is a prevalent cause of bacterial infections and biofilm formation associated with the blood stream and indwelling medical devices. Biofilms are made up of cells that are embedded in a protective polysaccharide matrix and exhibit an innate resistance to antibiotics, disinfectants and host defenses. Within bacteria, a biofilm matrix is able to resist antibiotics at concentrations from 1000 to 1500 times higher than are conventionally used. Infections associated with biofilms may result in longer hospital stays and an increased number of surgeries and deaths. Recurrent and chronic microbial infections are believed to be associated with biofilms. Novel compounds that inhibit biofilms but not growth, to avoid selection pressure for resistance, are needed.

Although the present invention has been described in terms of the foregoing preferred embodiments, this description has been provided by way of explanation only, and is not intended to be construed as a limitation of the invention. Those skilled in the art will recognize modifications of the present invention that might accommodate specific compounds or microbes. Such modifications do not necessarily depart from the spirit and scope of the invention.

I claim:

1. A composition for modulating bacterial biofilm virulence factors comprising gymnemic acids isolated from a botanical extraction of a plant of the genus Gymnema.

2. The composition of claim 1 wherein the gymnemic acids are isolated from a botanical extraction of Gymnema sylvestre.

3. The composition of claim 1 wherein the modulation of bacterial biofilm virulence factors comprises activating bacterial biofilm formation.

4. The composition of claim 3 wherein the gymnemic acids are isolated from a botanical extraction of Gymnema sylvestre.

5. The composition of claim 1 wherein the modulation of bacterial biofilm virulence factors comprises the modulation of Staphylococcus aureus biofilm virulence factors.

6. The composition of claim 5 wherein the gymnemic acids are isolated from a botanical extraction of Gymnema sylvestre.

7. The composition of claim 5 wherein the modulation of Staphylococcus aureus biofilm virulence factors comprises activating Staphylococcus aureus biofilm formation.

8. The composition of claim 7 wherein the gymnemic acids are isolated from a botanical extraction of Gymnema sylvestre.

9. A method for modulating bacterial biofilm virulence factors comprising the step of dispersing an effective amount of a mixture of gymnemic acids and an inert carrier.

10. The method of claim 9 wherein the step of modulating bacterial biofilm virulence factors comprises activating bacterial biofilm formation.

11. The method of claim 9 wherein the step of modulating bacterial biofilm virulence factors comprises modulating Staphylococcus aureus biofilm virulence factors.

12. The method of claim 10 wherein the step of activating bacterial biofilm formation comprises activating Staphylococcus aureus biofilm formation.

13. A composition for inhibiting bacterial biofilm formation comprising a polypeptide isolated from a botanical extraction of Gymnema sylvestre.

14. The composition of claim 13 wherein the polypeptide is gurmarin.

15. The composition of claim 13 wherein the inhibited bacterial biofilm comprises Staphylococcus aureus biofilm.

16. The composition of claim 15 wherein the polypeptide is gurmarin.

17. A method for isolating a biologically active polypeptide from a botanical source comprising the steps of producing an aqueous or aqueous/alcoholic extraction of the botanical source and isolating the polypeptide by isoelectric focusing separation.

18. A method for isolating gurmarin from Gymnema sylvestre plant material, the method comprising the steps of:
soaking dried leaf powders of the G. sylvestre plant in a water-alcohol mixture to produce an aqueous/alcoholic liquid extraction;
separating the liquid extraction into its constituent compounds by isoelectric focusing separation; and
selecting peptides having apparent molecular weight in the range of 4-5 kDa.

19. A method for treating object surfaces to inhibit the formation of bacterial biofilm comprising the step of dispersing an effective amount of a mixture of gurmarin and an inert carrier.

20. The method of claim 19, wherein the step of inhibiting the formation of bacterial biofilm comprises inhibiting Staphylococcus aureus biofilm.


SEQUENCE LISTING (19 sequences)

SEQ ID NO: 1 - protein, 35 aa, Gymnema sylvestre
QQCVKKDELCIPYYLDCCEPLECKKVNWWDHKCIG

SEQ ID NO: 2 - protein, 17 aa, Staphylococcus aureus
ALPQLSAGSNMQDYNMK

SEQ ID NO: 3 - protein, 11 aa, Staphylococcus aureus
LYDATQNIADK

SEQ ID NO: 4 - protein, 11 aa, Staphylococcus aureus
DMNGHALPLTK

SEQ ID NO: 5 - protein, 25 aa, Staphylococcus aureus
DGNFYQTNVDANGVNHGGSQMVQNK

SEQ ID NO: 6 - protein, 21 aa, Staphylococcus aureus
LSNGNTAGATGSSAAQIMAQR

SEQ ID NO: 7 - protein, 10 aa, Staphylococcus aureus
DGAYDIHFVK

SEQ ID NO: 8 - protein, 9 aa, Staphylococcus aureus
AQGLGAWGF

SEQ ID NO: 9 - protein, 9 aa, Staphylococcus aureus
VFYSFIDDK

SEQ ID NO: 10 - protein, 21 aa, Staphylococcus aureus
EYMSTLTYGFNGNVTGDDTGK

SEQ ID NO: 11 - protein, 7 aa, Staphylococcus aureus
YVQPDFK

SEQ ID NO: 12 - protein, 9 aa, Staphylococcus aureus
TILESPTDK

SEQ ID NO: 13 - protein, 16 aa, Staphylococcus aureus
VIFNNMVNQNWGPYDR

SEQ ID NO: 14 - protein, 10 aa, Staphylococcus aureus
AADNFLDPNK

SEQ ID NO: 15 - protein, 21 aa, Staphylococcus aureus
ASSLLSSGFSPDFATVITMDR

SEQ ID NO: 16 - protein, 13 aa, Staphylococcus aureus
DDYQLHWTSTNWK

SEQ ID NO: 17 - protein, 15 aa, Staphylococcus aureus
NMIKPGQELVVDKKQ

SEQ ID NO: 18 - protein, 15 aa, Staphylococcus aureus
DDPSQSTNVLGEAKK

SEQ ID NO: 19 - protein, 21 aa, Staphylococcus aureus
EDGNGVHVVKPGDTVNDIAKA